(19) United States
(12) Patent Application Publication
(10) Pub. No.: US 2026/0157623 A1
(43) Pub. Date: Jun. 11, 2026

(54) SYSTEMS AND METHODS FOR ASSESSING LENS DYSFUNCTION

(71) Applicant: Alcon Inc., Fribourg (CH)

(72) Inventors: George Hunter Pettit, Fort Worth, TX (US); John Alfred Campin, St. Cloud, FL (US); Joseph Weatherbee, San Clemente, CA (US)

(21) Appl. No.: 19/394,252

(22) Filed: Nov. 19, 2025

Related U.S. Application Data
(60) Provisional application No. 63/729,728, filed on Dec. 9, 2024.
(60) Provisional application No. 63/777,962, filed on Mar. 26, 2025.

(51) Int. Cl. A61B 3/10; A61B 3/00; A61B 3/14

(52) U.S. Cl. CPC A61B 3/102; A61B 3/0025; A61B 3/0041; A61B 3/145

(57) ABSTRACT

A method includes measuring an eye of a patient according to a plurality of imaging modalities to obtain a plurality of measurements, such as an OCT image, aberrometer measurement, or visible light image. The measurements are processed to obtain a characterization of lens scattering and a characterization of lens accommodation. A dysfunctional lens syndrome score is calculated for the lens according to the characterizations of lens scattering and accommodation and the dysfunctional lens syndrome score is output.

### TECHNICAL FIELD

The present disclosure relates generally to methods for the diagnosis of dysfunctional lens syndrome.

### BACKGROUND

Light received by the human eye, passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light.

Through age or disease, the crystalline lens may become cloudy, a condition known as a cataract. Cataracts are a readily treated by removing the crystalline lens and inserting an artificial lens, known as an intraocular lens (IOL). The IOL may be fabricated to additionally correct for aberrations of the patient's eye, such as astigmatism. Inasmuch as astigmatism is the result of asymmetry of the eye, the IOL must be aligned with the asymmetry of the eye in order to compensate for it. The IOL is therefore provided with markers, such as rows of dots at the perimeter of the IOL, which define an axis that may be used to align the IOL. The IOL may be implemented as a toric IOL, which includes spring-like arms, known as haptics, which hold the IOL in place within the capsular bag. In prior approaches, an imaging device, such as a digital marker microscope (DMM), is used to view the patient's eye during surgery. The image output by the imaging device has a reference axis superimposed thereon that corresponds to the desired orientation of the axis of the IOL.

Inasmuch as precise alignment of the IOL axis with the reference is desired, approaches for facilitating this alignment would greatly improve patient outcomes.

### BRIEF SUMMARY

The present disclosure relates generally to a system for characterizing the function of a lens of a patient's eye.

Particular embodiments disclosed herein provide a method including measuring the eye according to a plurality of imaging modalities to obtain a plurality of measurements; processing, by a computing system, the plurality of measurements to obtain a characterization of lens scattering and a characterization of lens accommodation; generating, by the computing system, a dysfunctional lens syndrome score for the lens according to the characterization of lens scattering and the characterization of lens accommodation; and outputting, by the computing system, the dysfunctional lens syndrome score. The plurality of imaging modalities may include one or both of an optical coherence tomography (OCT) device and an aberrometer and the plurality of measurements may include an OCT image and/or aberrometer measurements.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of the human eye.

FIG. 2 illustrates a multi-modal imaging device, in accordance with certain embodiments.

FIG. 3 illustrates a method for generating a dysfunctional lens syndrome score, in accordance with certain embodiments.

FIG. 4 illustrates a method for characterizing light scattering by a lens, in accordance with certain embodiments.

FIG. 5 illustrates a method for characterizing accommodation of an eye, in accordance with certain embodiments.

FIG. 6 illustrates a method for characterizing zonular integrity, in accordance with certain embodiments.

FIG. 7 illustrates an example computing device that implements, at least partly, one or more functionalities for generating a dysfunctional lens syndrome score, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Particular embodiments of the present disclosure provide an objective approach for diagnosing dysfunctional lens syndrome based on objective measurements of attributes of a lens such as scattering, accommodation, and zonular integrity.

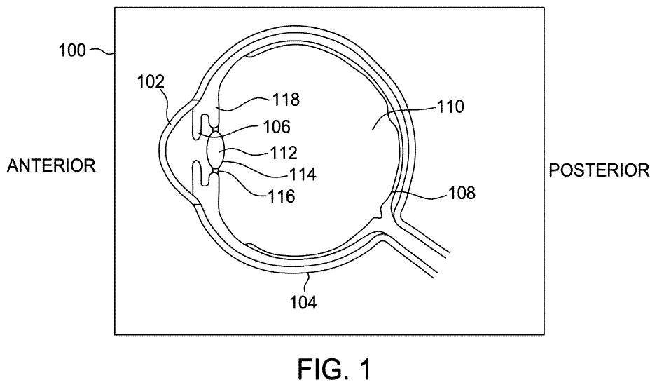

FIG. 1 is a diagram illustrating parts of the human eye 100 that may be understood with respect to the anterior side, through which light enters the eye, and the posterior side opposite the anterior side. At the anterior side of the eye 100, a thin transparent layer known as the cornea 102 is linked to the sclera 104, which forms the generally spherical wall of the eye 100. The cornea 102 and sclera 104 are connected by a ring called the limbus. The iris 106, the color of the eye, and an opening defined by it, the pupil, are positioned behind the cornea and are visible due to the cornea's 102 transparency. The retina 108 is formed on an interior surface of the sclera 104 opposite the cornea 102 and iris 16. The volume defined by the sclera 104 is occupied by the transparent jelly of the vitreous body 110.

The crystalline lens 112 is a transparent, biconvex structure in the eye that, along with the cornea 102, helps to refract light to be focused on the retina 108. The lens 112, by changing its shape, functions to change the focal distance of the eye so that it can focus on objects at various distances, thus allowing a sharp real image of the object of interest to be formed on the retina 108. This adjustment of the lens 112 is known as accommodation, and is similar to the focusing of a photographic camera via movement of its lenses.

The lens 112 is positioned behind the iris 106 in a capsular bag 114. The capsular bag 114 is attached at its perimeter to zonules 116. The zonules 116 are an array of fibers that attach the capsular bag 114 to the ciliary body 118. The ciliary body 118 includes a ring-shaped muscle that attaches the zonules 116 to the sclera 104 and which can contract or relax in order to change the shape of the lens 112.

Various diseases and disorders of the lens 112 may be treated with an IOL. By way of example, not necessarily limitation, an IOL may be used to treat cataracts, large optical errors in myopic (near-sighted), hyperopic (far-sighted), and astigmatic eyes, ectopia lentis, aphakia, pseudophakia, and nuclear sclerosis.

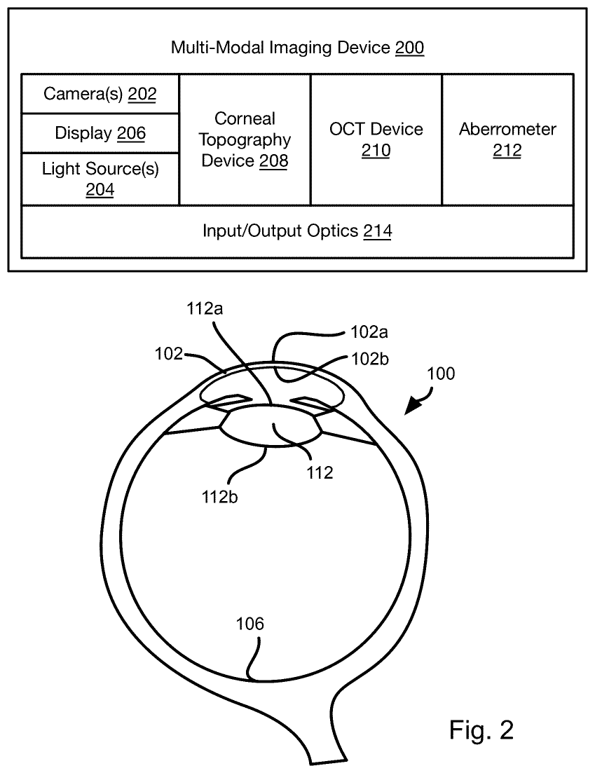

Referring to FIG. 2, imaging of the eye 100 may be performed using the illustrated multi-modal imaging device 200 or two or more separate imaging devices collectively performing the functions ascribed herein to the multi-modal imaging device 200.

The multi-modal imaging device 200 may include one or more cameras 202, such as a visible light camera. One or more light sources 204 may illuminate the eye 100 to facilitate capturing images with the one or more cameras 202. The one or more cameras 202 may be two cameras providing binocular vision. For example, the one or more cameras 202 and one or more light sources 204 may be implemented as the NGENUITY 3D VISUALIZATION SYSTEM provided by Alcon Inc. of Fort Worth Texas. The one or more cameras 202 may be used in combination with a display 206. The display 206 may display a fixation target that a patient may focus on in order to maintain the eye 100 in a desired orientation or to induce movement of the eye 100 as described below.

The one or more cameras 202 may capture reflections from the anterior surface 102a and posterior surface 102b of the cornea 102 and reflections from the anterior surface 112a and posterior surface 112b of the lens 112, the so-called Purkinje images.

The multi-modal imaging device 200 may include a corneal topography device 208.

The corneal topography device 208 measures the shape of the cornea 102 in order to estimate the diffractive power of the cornea 102 and any refractive error of the cornea 102, e.g., astigmatism. The corneal topography device 208 may measure the contours of the anterior and posterior surfaces 102a, 102b of the cornea 102 in order to perform the function thereof.

The multi-modal imaging device 200 may include an optical coherence tomography (OCT) device 210. The OCT device 210 obtains a volumetric image of the eye 100, including of one or both of the anterior and posterior chamber (region between the cornea 102 and the capsular bag 114) and the retina 108.

The multi-modal imaging device 200 may include an aberrometer 212, such as a wavefront aberrometer, that is configured to measure refractive error of the eye 100, including the combined refractive properties of the cornea 102, lens 112, and the axial length of the eye 100.

The various imaging devices of the multi-modal imaging device 200 may use input/output optics 214 to transmit light to the eye 100 and receive light reflected from the eye 100. The input/output optics 214 may include one or more lenses and/or beam splitters for routing light to the various imaging devices. Additionally or alternatively, each imaging device may have its own input/output optics.

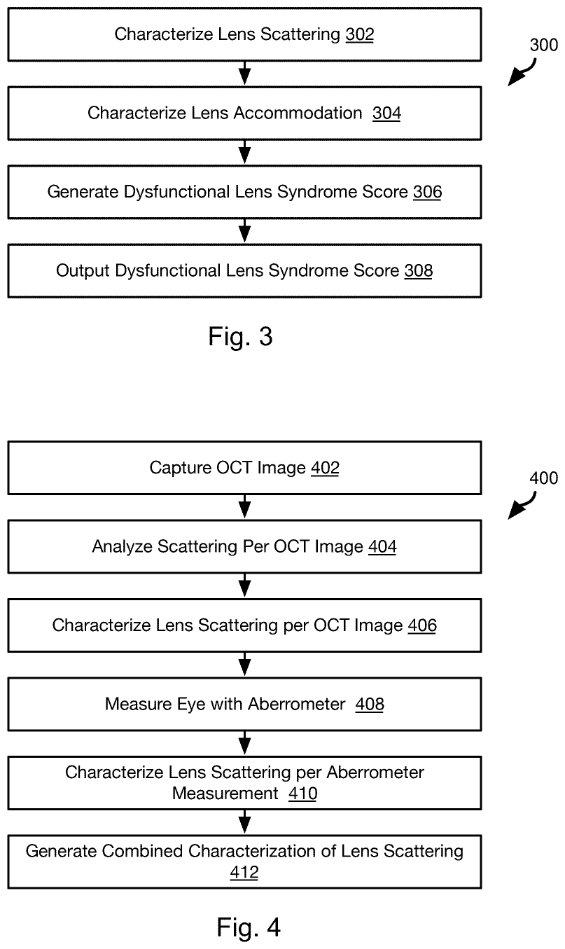

Referring to FIG. 3, various factors are considered when determining whether treatment of a lens 112 is needed, such as replacement of the lens 112 with an IOL. In general, a state of the lens 112 such that replacement is needed is referred to as “dysfunctional lens syndrome.” In prior practice, whether a lens 112 is dysfunctional and the degree of such dysfunction is the result of a very subjective evaluation by an ophthalmologist. Using the approach described herein, a more objective diagnosis of dysfunctional lens syndrome may be obtained based on objectively verifiable measurements.

A method 300 for diagnosing dysfunctional lens syndrome may include the illustrated steps, each of which will be described in greater detail below. The method 300 may include characterizing, at step 302, scattering of the lens 112 and characterizing, at step 304, accommodation of the lens 112. A dysfunctional lens syndrome score may be generated at step 306 and output at step 308.

FIG. 4 illustrates a method 400 for characterizing scattering of the lens 112 at step 302. The method 400 may include capturing, at step 402, an OCT image of the lens 112 using the OCT device 210. An OCT image captures an amount of scattering at points within the lens 112. The OCT image may be a cross-sectional image or a set of cross-sectional images defining a volumetric image of the lens 112. In an OCT image, a pixel, or volumetric pixel (voxel), records the amount of scattering at a particular point within the lens 112.

The method 400 may include analyzing, at step 404, scattering of the lens 112 according to the OCT image and characterizing, at step 406, scattering of the lens 112 according to the analysis of step 504. Step 404 may be performed using a machine learning model or a programmatic analysis of the pixel/voxel intensities of the OCT image to relate the detected scattering to a loss of visual acuity by the patient. Step 404 may include characterizing density of cataract tissue at points within the lens 112 and the location of the cataract tissue, e.g., distance from the optical axis of the lens 112.

Step 506 may include assigning a classification to the lens 112 according to the lens opacities classification system III (LOCS III). The type of each cataract indicated in the OCT image may be characterized based on location and other characteristics. Example types may include nuclear, cortical, posterior subcapsular, anterior subcapsular, diabetic snowflake, posterior polar, traumatic, congenital, polychromatic, or some other type. Step 406 may be performed using a machine learning model trained to perform this task based on the outputs of step 404 or directly from the OCT image. Step 406 may be performed using a programmatic method processing the outputs of step 404 or directly processing the OCT image.

Step 406 may include an aggregation of scattering indicated by pixels/voxels of the OCT image. For example, the output of step 406 may be obtained from estimated attenuation at various points on a wavefront passing through the lens 112, e.g., a total attenuation corresponding to scattering at points in the lens along the path of the point on the wavefront through the lens 112. The estimated attenuation for the various points may be aggregated, e.g., summed or weighted and summed, to obtain a characterization of scattering of the lens 112. Estimated attenuation may be weighted based on location: points on the wavefront closer to the optical axis of the lens 112 may be weighted more than points further away therefrom.

The method 400 may include measuring, at step 408, the eye 100 using the aberrometer 212. The aberrometer 212 may, for example, be a wavefront aberrometer. The aberrometer 212 measures refractive error of the eye 100, such as spherical error and astigmatism. The aberrometer 212 measures returning light after passing through the lens. The output of the aberrometer 212 therefore may indicate the presence of cataracts due to one or both of distortion of light caused by a cataract and a reduction in intensity of light returning after being incident on the retina 106. Additionally or alternatively, the output of the aberrometer 212 may indicate the presence of cataracts due to increased noise in the wavefront. For example, in the context of a Shack-Hartman wavefront sensor, the increase in noise may manifest as increase background levels between spots. In these and other embodiments, the noise increase in the wavefront can identify increase scattering due to the presence of cataracts. The aberrometer measurement may be in the form of one or more images, e.g., an image in which each pixel represents a phase error for reflected light corresponding to a point in an input wavefront and an image in which each pixel represents a reflected pixel intensity of reflected light corresponding to a point in the input wavefront.

The method 400 may include characterizing, at step 410, lens scattering according to the aberrometer measurement from step 408. Step 410 may include processing the aberrometer measurement using a machine learning model trained to output a characterization of lens scattering based on the aberrometer measurement, such as a classification (LOCS III) or type of any cataracts indicated by the aberrometer measurement. Step 410 may also include a programmatic processing of the aberrometer measurement to derive a characterization of scattering of the lens 112, such as a classification (LOCS III) or type of any cataracts indicated by the aberrometer measurement.

The characterization of scattering of step 410 may be based on an aggregation of attenuation and/or noise levels across points of a wavefront indicated by the aberrometer output. For example, attenuation at each point of a wavefront may be derived from an intensity of reflected light. The attenuation at a plurality of points may be summed or weighted and summed to obtain a characterization of scattering. The attenuation at each point may be weighted based on location: points on the wavefront closer to the optical axis of the lens 112 may be weighted more than points further away therefrom. As another example, noise levels at various points of the wavefront may be derived from background levels between the points of the wavefront.

The method 400 may include generating, at step 412, a combined characterization of lens scattering based on the outputs of steps 406 and 410. The output of step 412 may be used as the output of the method 400 and the characterization of lens scattering used at step 302 of the method 300. The combined characterization may be obtained by averaging the outputs of steps 406 and 410, e.g., an average of the cataract grades from steps 406 and 410. The combined characterization may include taking the minimum of steps 406 and 410, e.g., the lowest cataract grade, to provide higher confidence that the dysfunctional lens syndrome score is objectively correct. Cataract types may be combined by merging the cataract types identified at steps 406 and 410, e.g., the cataract types from step 406 plus any cataract types indicated by step 410 that were not indicated by step 406. Step 412 may include combining aggregations of attenuation from steps 406 and 410, such as by averaging, summing, or selecting the minimum or maximum aggregation.

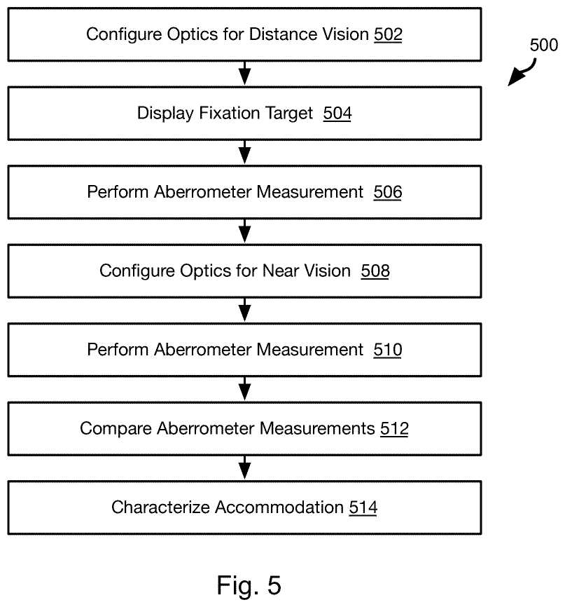

FIG. 5 illustrates a method 500 for characterizing accommodation. The method 500 may be used to implement step 304 of the method 300. The method 500 may include configuring, at step 502, optics for distance vision. The optics may be interposed between the one or more cameras 202 and the eye 100. The optics may be part of the input/output optics 214 or separate optics. Configuring the optics for distance vision may include configuring the optics such that a fixation target viewable by the eye 100 is at the optical equivalent of a distance at which the lens of the eye 100 is generally flattened, with little or no contraction of the ciliary muscles to curve the lens, e.g., at least 15 or at least 20 feet away. Step 502 may include configuring the optics to approximately (e.g., within 0.25 diopter) compensate for any astigmatism of the eye 100.

The method 500 may include displaying, at step 504, a fixation target, such as on the display 206, illuminating a static fixation target, or otherwise displaying a fixation target to the eye 100.

An aberrometer measurement may be performed at step 506 with the optics as configured at step 502 (e.g., distance vision) and the fixation target being displayed. The aberrometer measurement may be performed with the aberrometer 212. The aberrometer measurement may be replaced with any other approach for measuring refractive error of the eye 100.

The method 500 may include configuring, at step 508, the optics for near vision. For example, step 508 may include configuring the optics such that the fixation target is the optical equivalent of being closer to the eye 100, such as within 10 and 30 centimeters from the eye 100, the typical near point of a healthy, young eye being about 11 centimeters. Stated differently, the optics may be configured such that contraction of the ciliary muscles to curve the lens of the eye 100 is required but that a healthy eye should be able to perform sufficient contraction that there is no residual refractive error, e.g., hyperopic defocus.

Another aberrometer measurement may then be performed at step 510 with the optics configured per step 508 (e.g., near vision) and the fixation target displayed as at step 504. Step 510 may be performed sufficient time after performing step 508 that the eye 100 has plenty of time to adjust to the closer distance, such as at least 3, 5, or 10 seconds. Step 510 may be performed in the same manner as step 506. Steps 508 and 510 may be performed repeatedly for a range of distances such as at fixed intervals in terms of distance, diopters in the optics, or other increment.

The method 500 may include comparing, at step 512, the aberrometer measurements from steps 506 and 510. Accommodation may be characterized at step 514 based on the comparison. For example, if the difference between the distance vision (step 506) and near vision (step 510) is 10 diopters (D) and the actual distance is 10D, the accommodation is effective. As another example, if the difference between the distance vision (step 506) and near vision (step 510) is 0.5D, but the actual/expected difference is 5D, the accommodation is ineffective. In some embodiments, the effectiveness of accommodation may be based on a variation between the measured difference of distance/near vision and an actual or expected difference (e.g., a reference value). The larger the difference, the worse the accommodation of the eye 100. In some embodiments, accommodation of the eye may be characterized as a numerical value representing the observed variation. Where steps 508 and 510 are performed repeatedly, the near point of the eye 100 (e.g., the closest distance at which the eye 100 is able to effectively perform accommodation) may be identified based on the comparison, e.g., the distance at which the difference between the aberrometer measurement from an instance of step 510 and step 506 exceeds the reference value by some threshold, e.g., at least 0.25 diopters of spherical error.

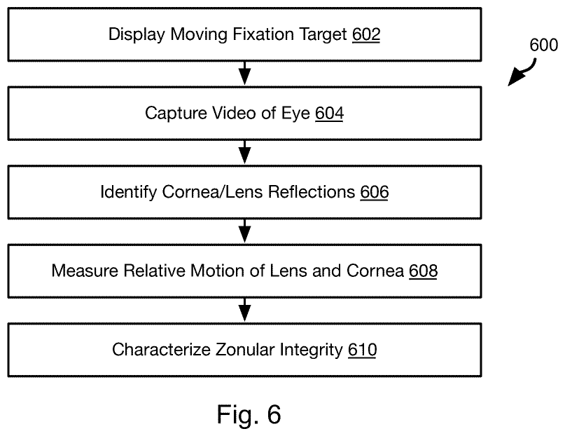

Referring to FIG. 6, in some embodiments, step 304 of the method 300 may include characterizing the function of the zonules 116, such as according to the illustrated method 600. If the zonules 116 become loose or tear, the ability of the eye 100 to adjust the shape of the lens 112 may be compromised. Likewise, uncontrolled movement of the lens 112 relative to the rest of the eye 100 may interfere with visual acuity during daily tasks.

The method 600 may include displaying, at step 602, a moving fixation target to the eye 100, such as on the display 206. The patient may be instructed to follow the fixation target with the eye 100. The velocity of the fixation target may be varied such that changes in the direction of movement the eye 100 will be induced.

The method may include capturing, at step 604, video of the eye 100 while performing step 602. The video may be analyzed at step 606 to identify reflections from the cornea 102 and lens 112. The reflections identified may be reflections of the fixation target, reflections of one or more light sources 204, or other reflections. The reflections may include reflections from the anterior and posterior surfaces 102a, 102b of the cornea 102 and reflections from the anterior and posterior surfaces 112a, 112b of the lens 112. The reflections may include the P1, P2, P3, and P4 Purkinje images. The P1 and P2 Purkinje images correspond to reflections from the anterior and posterior surfaces 102a, 102b, respectively, of the cornea 102. The P3 and P4 Purkinje images correspond to reflections from the anterior and posterior surfaces 112a, 112b, respectively, of the lens 112. Step 606 may include determining the locations of the reflections in two-dimensions in images of the video captured at step 604. In some embodiments, three-dimensional images are captured such that the three-dimensional locations of the reflections are identified.

At step 608, relative motion of the lens and cornea may be measured based on the reflections. For example, P1 and P2 may define a first line having a first angle relative to the optical axis of the eye 100. P3 and P4 may define a second line having a second angle relative to the optical axis of the eye 100. For a given time in the video (e.g., a frame or pair of frames captured at a time point), a difference between the first angle and the second angle may be calculated. Variation in the difference for the various frames of the video may likewise be determined. Variations in the difference between the first angle and the second angle over time may correspond to movement of the lens 112 relative to the cornea 102. The method 600 may therefore include characterizing zonular integrity at step 610 according to the variations such that the larger that variations in the difference, the lower the integrity of the zonules 116. In some embodiments, some statistical characterization of the variations (maximum, standard deviation, mean, etc.) may be used as the characterization of zonular integrity.

Referring again to FIG. 3, the characterization of lens scattering from step 302 (e.g., an output of the method 400) and the characterization of lens accommodation from step 304 (e.g., the outputs of the methods 500 and/or 600) may be combined to generate, at step 306, the dysfunctional lens syndrome score. For example, the output of the method 400 and the outputs of one or both of the methods 500, 600 may be summed, weighted and summed, or otherwise combined to obtain the dysfunctional lens syndrome score.

The dysfunctional lens syndrome score may be generated at step 306 and output at step 308. Outputting the dysfunctional lens syndrome may include outputting the dysfunctional lens syndrome score to a display device, storing it in a database, sending it as an email, text message, or other type of message, or outputting it as some other form of output.

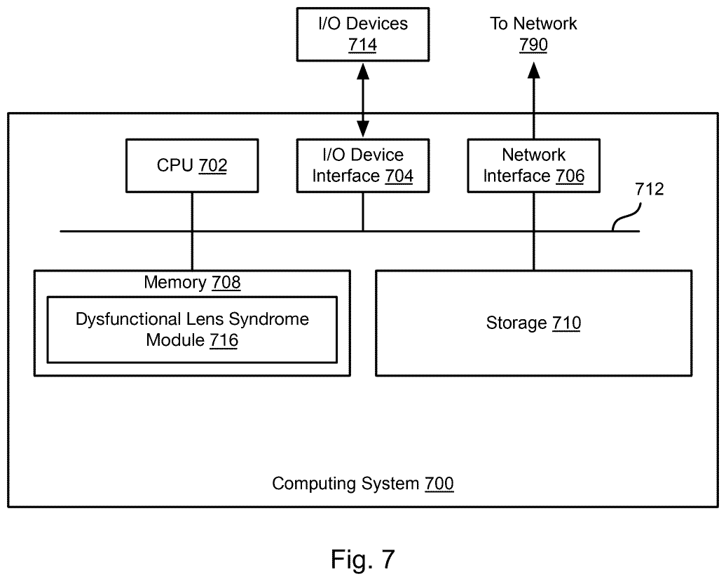

FIG. 7 illustrates an example computing system 700. The multi-modal imaging device 200 may have some or all of the attributes of the computing system 700.

As shown, computing system 700 includes a central processing unit (CPU) 702, one or more I/O device interfaces 704, which may allow for the connection of various I/O devices 714 (e.g., keyboards, displays, mouse devices, pen input, etc.) to computing system 700, network interface 706 through which computing system 700 is connected to network 790, a memory 708, storage 710, and an interconnect 712.

CPU 702 may retrieve and execute programming instructions stored in the memory 708. Similarly, CPU 702 may retrieve and store application data residing in the memory 708. The interconnect 712 transmits programming instructions and application data, among CPU 702, I/O device interface 704, network interface 706, memory 708, and storage 710. CPU 702 is included to be representative of a single CPU, multiple CPUs, a single CPU having multiple processing cores, and the like.

Memory 708 is representative of a volatile memory, such as a random access memory, and/or a nonvolatile memory, such as nonvolatile random access memory, phase change random access memory, or the like. As shown, memory 708 may store executable code implementing a dysfunctional lens syndrome module 716 which may be configured to perform the methods described above.

The computing system 700 may include storage 710, which may be non-volatile memory, such as a disk drive, solid state drive, or a collection of storage devices distributed across multiple storage systems.

### ADDITIONAL CONSIDERATIONS

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for evaluating a lens of an eye, the method comprising:
measuring the eye according to a plurality of imaging modalities to obtain a plurality of measurements, the plurality of imaging modalities includes an optical coherence tomography (OCT) device and the plurality of measurements including an OCT image;
processing, by a computing system, the plurality of measurements to obtain a characterization of lens scattering and a characterization of lens accommodation;
generating, by the computing system, a dysfunctional lens syndrome score for the lens according to the characterization of lens scattering and the characterization of lens accommodation; and
outputting, by the computing system, the dysfunctional lens syndrome score.

2. The method of claim 1, wherein the dysfunctional lens syndrome score is a combination of the characterization of lens scattering and the characterization of lens accommodation.

3. The method of claim 1, wherein the dysfunctional lens syndrome score is at least one of a sum or a weighted sum of the characterization of lens scattering and the characterization of lens accommodation.

4. The method of claim 1, wherein the plurality of imaging modalities further include at least one of:
a camera;
a corneal topography device; or
an aberrometer.

5. The method of claim 4, wherein the plurality of imaging modalities are incorporated into a multi-modal imaging device.

6. The method of claim 1, wherein:
the plurality of imaging modalities includes an aberrometer and the plurality of measurements includes an aberrometer measurement; and
processing the plurality of measurements to obtain the characterization of lens scattering comprises processing the aberrometer measurement.

7. The method of claim 1, wherein:
the plurality of imaging modalities includes an aberrometer; and
processing the plurality of measurements to obtain the characterization of lens accommodation comprises measuring, using the aberrometer, a change in refractive error of the eye in response to a change in distance to a fixation target.

8. The method of claim 1, wherein:
the plurality of imaging modalities includes one or more visible light cameras and a display device; and
the method further comprises:
displaying, by the computing system, a moving fixation target on the display device;
capturing video images of the eye while displaying the moving fixation target using the one or more visible light cameras;
identifying, by the computing system, reflections from anterior and posterior surfaces of the lens in the video images;
identifying, by the computing system, reflections from anterior and posterior surfaces of a cornea of the eye in the video images;
measuring, by the computing system, relative motion between the lens and the cornea according to the reflections from anterior and posterior surfaces of the lens and the reflections from anterior and posterior surfaces of a cornea; and
characterizing, by the computing system, integrity of zonules of the eye according to the relative motion.

9. A system for evaluating a lens of an eye, the system comprising:
a plurality of imaging devices configured to measure the eye according to a plurality of imaging modalities to obtain a plurality of measurements, the plurality of imaging devices including an optical coherence tomography (OCT) device and the plurality of measurements including an OCT image;
a computing system including one or more processing devices and one or more memory devices storing executable code that, when executed by the one or more processing devices, causes the one or more processing devices to:
processing, by a computing system, the plurality of measurements to obtain a characterization of lens scattering and a characterization of lens accommodation;
generating, by the computing system, a dysfunctional lens syndrome score for the lens according to the characterization of lens scattering and the characterization of lens accommodation; and
outputting, by the computing system, the dysfunctional lens syndrome score.

10. The system of claim 9, wherein the dysfunctional lens syndrome score is a combination of the characterization of lens scattering and the characterization of lens accommodation.

11. The system of claim 9, wherein the dysfunctional lens syndrome score is at least one of a sum or a weighted sum of the characterization of lens scattering and the characterization of lens accommodation.

12. The system of claim 9, wherein the plurality of imaging devices further include at least one of:
a camera;
a corneal topography device; or
an aberrometer.

13. The system of claim 12, wherein the plurality of imaging devices are incorporated into a multi-modal imaging device.

14. The system of claim 9, wherein:
the plurality of imaging devices includes an aberrometer and the plurality of measurements includes an aberrometer measurement; and
the executable code, when executed by the one or more processing devices, causes the one or more processing devices to process the plurality of measurements to obtain the characterization of lens scattering by processing the aberrometer measurement.

15. The system of claim 9, wherein:
the plurality of imaging modalities includes an aberrometer; and
the executable code, when executed by the one or more processing devices, causes the one or more processing devices to process the plurality of measurements to obtain the characterization of lens accommodation by measuring, using the aberrometer, a change in refractive error of the eye in response to a change in distance to a fixation target.

16. The system of claim 9, wherein:
the plurality of imaging devices includes one or more visible light cameras and a display device; and
the executable code, when executed by the one or more processing devices, causes the one or more processing devices to:
capture video images of the eye using the one or more visible light cameras while displaying a moving fixation target using the display device;
identify reflections from anterior and posterior surfaces of the lens in the video images;
identify reflections from anterior and posterior surfaces of a cornea of the eye in the video images;
measure relative motion between the lens and the cornea according to the reflections from anterior and posterior surfaces of the lens and the reflections from anterior and posterior surfaces of a cornea; and
characterize integrity of zonules of the eye according to the relative motion.

17. A method for evaluating a lens of an eye, the method comprising:
measuring the eye according to a plurality of imaging modalities to obtain a plurality of measurements, the plurality of imaging modalities includes an aberrometer and the plurality of measurements includes an aberrometer measurement;
processing, by a computing system, the plurality of measurements to obtain a characterization of lens scattering and a characterization of lens accommodation;
generating, by the computing system, a dysfunctional lens syndrome score for the lens according to the characterization of lens scattering and the characterization of lens accommodation; and
outputting, by the computing system, the dysfunctional lens syndrome score.

18. The method of claim 17, wherein the dysfunctional lens syndrome score is a combination of the characterization of lens scattering and the characterization of lens accommodation.

19. The method of claim 17, wherein the dysfunctional lens syndrome score is at least one of a sum or a weighted sum of the characterization of lens scattering and the characterization of lens accommodation.

20. The method of claim 17, wherein the plurality of imaging modalities further include at least one of:
a camera;
a corneal topography device; and
an optical coherence tomography (OCT) device.

21. The method of claim 20, wherein the plurality of imaging modalities are incorporated into a multi-modal imaging device.

22. The method of claim 17, wherein processing the plurality of measurements to obtain the characterization of lens accommodation comprises measuring, using the aberrometer, a change in refractive error of the eye in response to a change in distance to a fixation target.

23. The method of claim 17, wherein:
the plurality of imaging modalities includes one or more visible light cameras and a display device; and
the method further comprises:
displaying, by the computing system, a moving fixation target on the display device;
capturing video images of the eye while displaying the moving fixation target using the one or more visible light cameras;
identifying, by the computing system, reflections from anterior and posterior surfaces of the lens in the video images;
identifying, by the computing system, reflections from anterior and posterior surfaces of a cornea of the eye in the video images;
measuring, by the computing system, relative motion between the lens and the cornea according to the reflections from anterior and posterior surfaces of the lens and the reflections from anterior and posterior surfaces of a cornea; and
characterizing, by the computing system, integrity of zonules of the eye according to the relative motion.

24. A system for evaluating a lens of an eye, the system comprising:
a plurality of imaging devices configured to measure the eye according to a plurality of imaging modalities to obtain a plurality of measurements, the plurality of imaging devices includes an aberrometer and the plurality of measurements includes an aberrometer measurement;
a computing system including one or more processing devices and one or more memory devices storing executable code that, when executed by the one or more processing devices, causes the one or more processing devices to:
processing, by a computing system, the plurality of measurements to obtain a characterization of lens scattering and a characterization of lens accommodation;
generating, by the computing system, a dysfunctional lens syndrome score for the lens according to the characterization of lens scattering and the characterization of lens accommodation; and
outputting, by the computing system, the dysfunctional lens syndrome score.

25. The system of claim 24, wherein the dysfunctional lens syndrome score is a combination of the characterization of lens scattering and the characterization of lens accommodation.

26. The system of claim 24, wherein the dysfunctional lens syndrome score is at least one of a sum or a weighted sum of the characterization of lens scattering and the characterization of lens accommodation.

27. The system of claim 24, wherein the plurality of imaging devices further include one or more of:
a camera;
a corneal topography device; or
an optical coherence tomography (OCT) device.

28. The system of claim 27, wherein the plurality of imaging devices are incorporated into a multi-modal imaging device.

29. The system of claim 24, wherein the executable code, when executed by the one or more processing devices, causes the one or more processing devices to process the plurality of measurements to obtain the characterization of lens accommodation by measuring, using the aberrometer, a change in refractive error of the eye in response to a change in distance to a fixation target.

30. The system of claim 24, wherein:
the plurality of imaging devices includes one or more visible light cameras and a display device; and
the executable code, when executed by the one or more processing devices, causes the one or more processing devices to:
capture video images of the eye using the one or more visible light cameras while displaying a moving fixation target using the display device;
identify reflections from anterior and posterior surfaces of the lens in the video images;
identify reflections from anterior and posterior surfaces of a cornea of the eye in the video images;
measure relative motion between the lens and the cornea according to the reflections from anterior and posterior surfaces of the lens and the reflections from anterior and posterior surfaces of a cornea; and
characterize integrity of zonules of the eye according to the relative motion.
